(19) United States
(12) Patent Application Publication
(10) Pub. No.: US 2005/0203338 A1
(43) Pub. Date: Sep. 15, 2005

(54) Endoscope with fiber optic transmission of digital video

(76) Inventors: Lucien A. Couvillon JR., Concord, MA (US); Michael S. Banik, Bolton, MA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/798,587

(22) Filed: Mar. 10, 2004

(51) Int. Cl. A61B 1/04

(52) U.S. Cl. 600/109; 600/182; 600/118

(57) ABSTRACT

A video endoscope in which the imaging chip at the distal tip is connected to the proximal connector and operator console by an optical fiber. Video data (preferably digital) is impressed on the optical fiber by a pulse-code modulator (PCM) circuit and light emitter with lensing. A small, inexpensive, flexible optical fiber in the endoscope body conveys the PCM optical data to the receiving circuit, preferably via an optical connector at the proximal end of the endoscope, and to decoding and display circuits in the operator console. The fiber optic transmission overcomes the difficulty of electrical transmission of wide band (digital video) signals via miniature cables, which can be expensive, bulky, inflexible, susceptible to noise and interference, and constitute a potential electrical safety risk by leakage currents. The PCM circuits may be integrated into the camera chip.

### FIELD OF THE INVENTION

The present invention relates to medical devices, and in particular to imaging endoscopes.

### BACKGROUND OF THE INVENTION

Many medical interventional procedures are dependent on endoscopes to deliver diagnostic and therapeutic catheters to gastroenterological, alimentary, pulmonary, urological, reproductive, biliary, and other locations throughout the body. In a fiber optic endoscope, both the illumination channel and the imaging channel may be made of a bundle of optical fibers. The illumination channel is coupled to a light source to illuminate an internal body cavity of a patient, and the imaging channel transmits an image created by a lens at the distal end of the endoscope to a connected camera unit or display device.

As imaging electronics, especially miniature CCD or CMOS image sensors, have advanced, endoscope designers have moved to placing imaging arrays at the distal tip of the endoscope. Such designs produce higher resolution images, because they provide a much larger pixel count than fiber optic bundles. In video endoscopes (i.e., endoscopes with a video camera chip at the distal end), the video camera chip requires two-way electrical connection, for power and control upstream, and video downstream. CMOS imaging chips are emerging as preferable to CCD chips because they are simple, their cost is decreasing, they are inherently all-digital, and they provide comparable or superior image quality.

In video endoscopes, the connection of the CMOS camera chip to the operator console is a key design problem because the electronic video signal has a very high bandwidth and may be at low voltage levels. Electrical power is easier to handle than the video signal transmission, because the upstream camera current requirements are small, but the downstream video bandwidth is large. Miniature electrical cables are suitable for the power supply and any control logic but the video cables may have to be coaxial or twisted-pair to handle the high bandwidth and the need for an accurate impedance match, and may have to be shielded to guard against electromagnetic susceptibility or emission interference standards. Such systems can be expensive, bulky, inflexible, susceptible to noise and interference, and might even contribute to a potential electrical safety risk due to leakage currents.

The size of the endoscope is an important factor, because in many endoscopic procedures the area for maneuvering the endoscope is limited by the working channel diameter. Further limitations regarding the areas that are accessible to the endoscope are due to the physical constraints caused by the size and stiffness of the endoscope. Furthermore, larger endoscopes may contribute to the discomfort of the patient, making the endoscopic procedure painful and requiring sedation.

The present invention is directed to an apparatus that overcomes the foregoing and other disadvantages. More specifically, the present invention is directed to an endoscope in which a wide band video data stream is impressed on an optical fiber by a pulse-code modulation, thus allowing for the production of a smaller, simpler, and less expensive, electrically isolated endoscope.

### SUMMARY OF THE INVENTION

The present invention is directed to a video endoscope in which the imaging chip at the distal tip communicates with the proximal connector and operator console by means of electrical signals impressed on an optical fiber. Electronic video data are encoded upon the optical fiber by a pulse-code modulator (PCM) circuit and light emitter with lensing. In one embodiment, the video data are digital. A small, inexpensive, flexible optical fiber in the endoscope body thus conveys the PCM optical data to a receiving circuit, preferably via an optical connector at the proximal end of the endoscope, or by a proximal optical-to-electronic video chip and electrical connector, and to decoding and display circuits in the operator console. It will be appreciated that fiber optic transmission overcomes the difficulty of electrical transmission of wide band digital video signals via miniature cables, which can be expensive, bulky, inflexible, susceptible to noise and interference, and constitute a potential electrical safety risk by leakage currents.

In accordance with another aspect of the invention, the PCM circuits and optical interface circuits may be integrated into the camera chip. It will be appreciated that this is a relatively straightforward design for CMOS chips, most of which already include on-board analog-to-digital converter circuits and line drivers, and electro-optical functions. Other commonly available components may also be utilized. In one embodiment, the light emitter that is used is similar to devices used in consumer CD and DVD readers and recorders. The optical fiber and connectors may be of the types used in telecommunications. The light detector which is utilized converts the optical signals to PCM logic signals for conventional decoding. High-bandwidth components suitable for the real time, VGA-resolution video stream may be utilized which are of the types used in computers, consumer electronics and telecommunications.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows an endoscope system formed in accordance with the present invention;

FIG. 2 shows the components at the distal end of the endoscope system of FIG. 1; and

FIG. 3 shows the components at the console end of the endoscope of FIG. 1.

### DETAILED DESCRIPTION OF THE PREFERRED EMBODIMENT

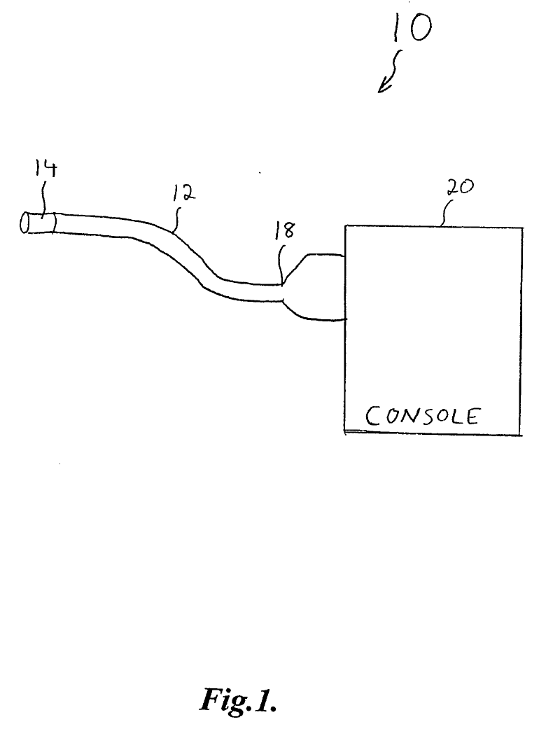

FIG. 1 is a diagram of an endoscope system 10 that is formed in accordance with the present invention. The endoscope system 10 includes a flexible catheter body 12 having a distal end 14. The catheter body 12 also includes a proximal end 18 which is coupled to a console 20. The console 20 provides electrical connections for the catheter body, such that the image signals can be received and processed, as will be described in more detail below.

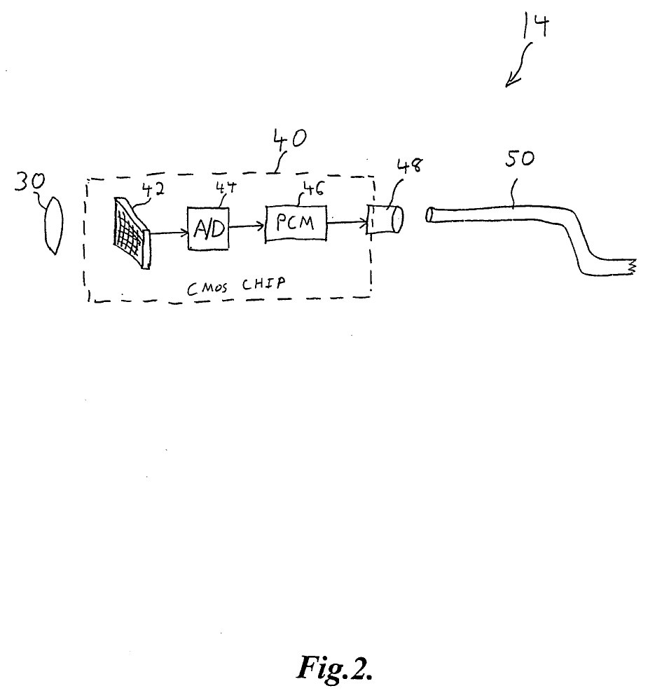

FIG. 2 shows the components of the distal tip 14 of the endoscope system 10. The distal tip 14 includes an optical lens 30, a CMOS chip 40, a photo diode 48, and a transmission fiber 50. The lens 30 may be a distal objective lens, and may also represent a lens system. The lens 30 is placed in front of the CMOS chip 40. The CMOS chip 40 includes an imaging array 42, an analog-to-digital converter 44, and a pulse-code modulator (PCM) circuit 46. As will be described in more detail below, the photo diode 48 transmits the signals from the CMOS chip 40 to the transmission fiber 50 which carries the video signals to the operator console 20.

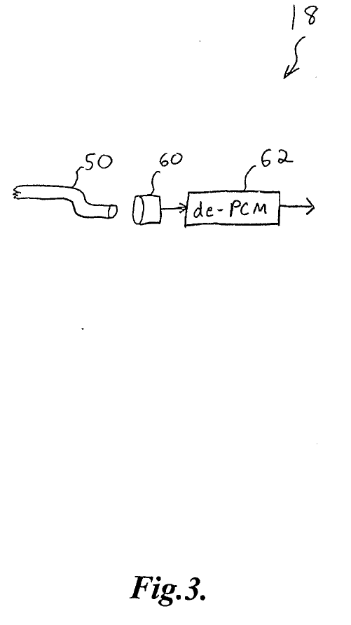

The imaging chip 40 at the distal tip 14 is thus connected to the proximal connector and operator console 20 by the optical fiber 50. Video data (preferably digital) is impressed on the optical fiber 50 by the pulse code modulator circuit 46 and the light emitter 48 with lensing. The small, inexpensive, flexible optical fiber 50 in the endoscope body 12 conveys the PCM optical data to a receiving circuit in the console 20, which will be described in more detail below with reference to FIG. 3.

FIG. 3 shows the components at the proximal end 18 and console 20 of the video endoscope system 10. As shown in FIG. 3, the transmission fiber 50 transmits signals to a photo detector 60, which is coupled to a pulse-code demodulator circuit 62. In other words, the transmission fiber 50 conveys the PCM optical data to the receiving circuit, preferably via an optical connector at the proximal end 12, and to decoding circuitry 62 and to display circuitry (not shown) in the operator console 20.

It will be appreciated that fiber optic transmission overcomes the difficulty of electrical transmission of wide band (digital video) signals via miniature cables, which can otherwise be expensive, bulky, inflexible, susceptible to noise and interference, and constitute a potential electrical safety risk by leakage currents. It will be further appreciated that the PCM circuits may be integrated into the camera chip, and that this is a straightforward process for conventional CMOS chips, most of which already include on-board analog-to-digital converter circuits and line drivers. The light emitter that is utilized may be of the type that is readily available as a component that is commonly used in consumer CD and DVD readers and recorders. The optical fiber and connectors that are used may also be of the type that are readily available as being commonly used in telecommunications. Conventional decoding may be utilized for the light detector converting the optical signals to PCM logic signals. High bandwidth components that are suitable for the real time, VGA-resolution video stream may be utilized that are also commonly available due to continuing advancements in the consumer electronics and telecommunications fields.

The use of the transmission fiber is advantageous in that it provides for total electrical isolation. The operation of the device may be further simplified by using certain known techniques. For example, it is relatively simple to drive the PCM onto the transmission fiber by using a fast photo diode (e.g., blue or IR wave lengths). It is also relatively simple to photo detect the signals at the proximal end using a simple fast photo detector. Due to the nature of the fiber transmissions, the signals are relatively immune to RFI, thus not requiring an RF shield or coaxial cables, thus making the system less complex and less expensive. The inclusion of the digital-to-PCM encoding on the CMOS imaging chip makes the system more compact and less expensive. In one embodiment, the photo diode may also be included on the CMOS chip. The utilization of “off-the-shelf” components from the CD and DVD recorder industry further reduces the expense and complexity of the system, as does the utilization of off-the-shelf fiber optic cable from the telecom industry.

While the preferred embodiment of the invention has been illustrated and described, it will be appreciated that various changes can be made therein without departing from the spirit and scope of the invention.

1. A video endoscope system, comprising:
an endoscope with a distal end and a proximal end;
an imaging chip located at the distal end of the endoscope; and
an optical fiber, the optical fiber being used for transmitting signals from the imaging chip.

2. The system of claim 1, further comprising an operator console, wherein the video chip is coupled through the optical fiber to the operator console.

3. The system of claim 1, further comprising an encoder circuit, wherein the encoder circuit encodes the signals from the imaging chip in the form of video data that is impressed on the optical fiber.

4. The system of claim 3, wherein the encoder circuit comprises a pulse-code modulator.

5. The system of claim 1, further comprising a light emitter, wherein the light emitter is utilized for impressing video data on the optical fiber.

6. The system of claim 5, wherein the light emitter is a photo diode.

7. The system of claim 5, further comprising lensing that is coupled to the light emitter.

8. The system of claim 1, further comprising a receiving circuit for receiving signals from the optical fiber.

9. The system of claim 8, further comprising an optical connector at the proximal end of the endoscope, the optical connector being used to couple the optical fiber to the receiving circuits.

10. The system of claim 8, wherein the receiving circuit comprises a decoder for decoding signals received from the optical fiber.

11. The system of claim 10, wherein the decoder comprises a pulse-code demodulator.

12. A video endoscope, comprising:
an encoding circuit; and
an optical fiber, wherein video data is impressed on the optical fiber by the encoding circuit.

13. The endoscope of claim 12, wherein the encoding circuit comprises a pulse-code modulator.

14. The endoscope of claim 12, wherein the encoding circuit comprises a light emitter.

15. The endoscope of claim 14, wherein the light emitter is a photo diode that is used for creating the signals that are impressed on the optical fiber.

16. An endoscope system, comprising:
an imaging chip located at the distal end of the endoscope;
an operator console; and
an optical fiber, wherein the imaging chip is coupled through the optical fiber to the operator console.

17. They system of claim 16, further comprising an encoder circuit, wherein the encoder circuit encodes video data from the imaging chip that is transmitted through the optical fiber.

18. The system of claim 17, wherein the encoder circuit comprises a pulse-code modulator.

19. The system of claim 17, further comprising a light emitter, wherein the light emitter is coupled to the encoder circuit and is utilized for creating the signals that are transmitted through the optical fiber.

20. The system of claim 19, wherein the light emitter is a photo diode.

21. The system of claim 16, further comprising a receiving circuit in the operator console for receiving signals from the optical fiber.

22. The system of claim 21, further comprising an optical connector at the proximal end of the endoscope, the optical connector being used to couple the optical fiber to the receiving circuit.

23. The system of claim 21, wherein the receiving circuit comprises a decoder for decoding signals received from the optical fiber.

24. The system of claim 23, wherein the decoder comprises a pulse-code demodulator.
